(19) United States
(12) Patent Application Publication
(10) Pub. No.: US 2026/0249080 A1
(43) Pub. Date: Aug. 27, 2026

(54) SYSTEMS AND METHODS FOR REGULATING INFLAMMATION AND IMMUNE RESPONSES USING BAROREFLEX ACTIVATION THERAPY

(71) Applicant: CVRx, Inc., Minneapolis, MN (US)

(72) Inventors: Philip B. Adamson, Austin, TX (US); Paul Pignato, Jacksonville, FL (US); Tucker Stuart, North Saint Paul, MN (US); Bart Carey, Maplewood, MN (US); Seth J. Wilks, Valparaiso, IN (US); Y-Lan Nguyen Cruea, Newport Beach, CA (US)

(21) Appl. No.: 19/551,267

(22) Filed: Feb. 26, 2026

Related U.S. Application Data
(60) Provisional application No. 63/764,324, filed on Feb. 27, 2025.

(51) Int. Cl. A61N 1/36; A61N 1/372

(52) U.S. Cl. CPC A61N 1/36139; A61N 1/36057; A61N 1/36071; A61N 1/36114; A61N 1/3615; A61N 1/36171; A61N 1/36175; A61N 1/37217; A61N 1/37235

(57) ABSTRACT

This disclosure presents systems and methods for baroreflex activation therapy (BAT) to regulate inflammation by modulating autonomic nervous system activity. Electrical stimulation is delivered to one or more baroreceptors, and real-time physiological signals are monitored, including inflammatory biomarkers (e.g., TNF-α, IFN-γ, IL-1, IL-6, IL-12, CRP) and autonomic parameters such as heart rate variability (HRV). A closed-loop feedback module analyzes biomarker fluctuations and autonomic balance changes and dynamically modifies stimulation parameters, including amplitude, pulse width, frequency, and duty cycle, to suppress systemic inflammation and restore autonomic balance. In certain embodiments, multi-channel stimulation architectures and AI-driven algorithms or machine learning models analyze biomarker trends and historical patient response patterns to optimize therapy. The disclosed BAT systems and methods may be used to treat inflammatory diseases, autoimmune disorders, cancer progression, chronic inflammatory pain conditions, arrhythmias associated with inflammation, and metabolic dysfunction.

### CLAIM OF PRIORITY

This non-provisional patent document claims the benefit of priority under 35 U.S.C. § 119(e) to U.S. Provisional Patent Application Ser. No. 63/764,324, Entitled “systems and Methods for Regulating Inflammation AND IMMUNE RESPONSES USING BAROREFLEX ACTIVATION THERAPY,” filed on Feb. 27, 2025, which is incorporated herein by reference in its entirety.

### TECHNICAL FIELD

This patent document relates generally to neuromodulation therapies and, more specifically, to systems and methods for modulating inflammation and immune responses.

### BACKGROUND

Baroreflex activation therapy (BAT) is a neuromodulation technique that regulates autonomic nervous system (ANS) activity by stimulating baroreceptors. Clinically validated for hypertension and heart failure, BAT mitigates sympathetic overactivity, a key factor in these disease progressions. By restoring autonomic balance, BAT has been shown to lower blood pressure and enhance cardiac function.

### OVERVIEW

The present inventors recognize the fundamental role of the ANS in regulating inflammation and immune responses. Sympathetic nervous system (SNS) dysregulation can contribute to various disease states, including inflammatory and autoimmune disorders, cancer, chronic pain, arrhythmias associated with inflammation, and Type II diabetes and metabolic dysfunction. Chronic sympathetic overactivity promotes elevated cytokine levels, immune cell overactivation, and neuroimmune dysfunction, whereas parasympathetic activation exerts anti-inflammatory effects via the cholinergic anti-inflammatory pathway.

Current treatments rely on pharmacological interventions such as immunosuppressants, biologic drugs (e.g., TNF inhibitors, IL-1, IL-6, and IL-12 blockers), NSAIDs, corticosteroids, and opioids. These therapies often provide only symptomatic relief, target specific inflammatory pathways, and may lead to side effects or long-term tolerance issues. Moreover, they do not address the underlying autonomic dysfunction driving systemic inflammation.

This disclosure describes BAT-based systems and methods to modulate ANS activity for treating inflammatory diseases, autoimmune disorders, cancer, chronic pain, arrhythmias, and Type II diabetes. BAT stimulates baroreceptors, such as those in the carotid sinus, via a pulse generator, reducing SNS activity, enhancing parasympathetic tone, and suppressing systemic inflammation. By restoring sympathovagal balance, BAT offers a non-pharmacological alternative or adjunct to conventional treatments, potentially reducing reliance on medications with adverse effects or limited long-term efficacy.

In one embodiment, the BAT system comprises an implantable pulse generator (IPG), a flexible lead with one or more electrodes, and an external programmer and controller for therapy adjustments. The IPG can be implanted subcutaneously near the clavicle, with the lead positioned along the carotid sinus for direct baroreceptor stimulation. The external programmer enables therapy adjustments by clinicians or industry professionals, allowing modifications to stimulation intensity, frequency, and duty cycle to optimize therapy for individual patient responses.

Some embodiments incorporate real-time closed-loop control mechanisms with feedback sensors, including heart rate variability (HRV) monitors to track sympathetic nervous system (SNS) and parasympathetic activity, blood pressure monitors to assess cardiovascular response, inflammatory biomarker sensors to detect cytokines such as TNF-α, IFN-γ, IL-1, IL-6, IL-12, and C-reactive protein (CRP) to gauge inflammation, and electromyography (EMG) sensors to analyze neuromuscular responses and autonomic changes. These biometric feedback sensors enable real-time closed-loop control, allowing autonomous therapy adjustments based on patient physiology.

The BAT system can be particularly beneficial for treating autoimmune and inflammatory diseases, including rheumatoid arthritis, systemic lupus erythematosus, Crohn's disease, ulcerative colitis, and long COVID-related inflammatory conditions. It can also benefit cardiovascular conditions, such as inflammation-associated arrhythmias, myocarditis, and post-surgical atrial fibrillation, as well as chronic inflammatory pain syndromes, neuropathic pain, arthritis, and fibromyalgia.

Artificial intelligence (AI)-driven algorithms may be integrated into the BAT system to analyze biomarker trends, autonomic function, and patient response patterns for predictive therapy optimization. Machine learning models can dynamically adjust stimulation parameters in response to real-time physiological feedback, ensuring personalized and adaptive treatment.

A further embodiment can include a non-invasive BAT system that provides temporary neuromodulation via external stimulation for patients who may not require permanent implantation. The system may also feature multi-channel stimulation, enabling precise baroreceptor activation at varying intensities, frequencies, and duty cycles tailored to inflammatory, autoimmune, and pain-related conditions. Additionally, wearable patient monitoring interfaces may allow remote therapy adjustments and real-time patient tracking, improving long-term disease management and treatment personalization.

The disclosed BAT systems and methods provide an adaptable and effective approach to treating conditions driven by sympathetic overactivity and chronic inflammation. By leveraging neuromodulation as a systemic anti-inflammatory strategy, BAT extends beyond hypertension and heart failure applications into broader immune response regulation. This positions BAT as a transformative therapy for inflammatory diseases, autoimmune disorders, cancer, chronic pain (particularly arthritis and joint pain), and inflammation-associated arrhythmia.

By offering a personalized, non-pharmacological treatment option, BAT reduces dependence on conventional drug therapies, which often have side effects and long-term tolerance issues. Instead, BAT presents a sustainable alternative targeting autonomic dysfunction and immune dysregulation at its root rather than merely managing symptoms.

Furthermore, integrating adaptive neuromodulation, biosensor feedback, and machine learning-driven optimizations can ensure highly precise and individualized therapy. By continuously analyzing real-time physiological data, inflammatory markers, and autonomic function, BAT can dynamically adjust stimulation parameters to maximize therapeutic efficacy and patient safety. This makes BAT a scalable, patient-centric intervention ideally suited for autonomic dysfunction and chronic inflammation-related conditions, ultimately improving clinical outcomes and quality of life for affected patients.

These and other examples and features of the disclosed systems and methods will be described in greater detail in the following Detailed Description. This Overview is intended to present non-limiting examples of the disclosed subject matter and is not meant to serve as an exhaustive or exclusive explanation. Instead, the Detailed Description below provides further information regarding the design, implementation, and applications of the disclosed systems and methods.

### BRIEF DESCRIPTION OF THE DRAWINGS

In the drawings, like numerals are used to describe similar features and components throughout the several views. The drawings illustrate generally, by way of example, but not by way of limitation, various system and method embodiments discussed in this patent document.

FIG. 1 illustrates a schematic diagram of a BAT system, including an IPG, a lead with one or more electrodes, feedback sensors, an external programmer, and a patient interface, as constructed in accordance with at least one embodiment.

FIG. 2 illustrates a schematic diagram showing the subcutaneous placement of an IPG near the clavicle and the positioning of a lead along the carotid artery, as constructed in accordance with at least one embodiment.

FIG. 3 illustrates a schematic diagram showing how real-time feedback sensors (e.g., HRV, biomarker sensors, EMG) interact with an IPG and an external programmer to adjust stimulation parameters dynamically, as constructed in accordance with at least one embodiment.

FIG. 4 illustrates a tiered flowchart showing how BAT stimulation can affect the ANS, reducing SNS activity, enhancing vagal tone, decreasing norepinephrine and pro-inflammatory cytokines, and modulating pain reception, as constructed in accordance with at least one embodiment.

FIG. 5 illustrates a schematic diagram showing how AI algorithms can process patient data, predict responses, and optimize stimulation settings via machine learning models, as constructed in accordance with at least one embodiment.

FIG. 6 illustrates a step-by-step implantation procedure for a BAT system, as constructed in accordance with at least one embodiment.

FIG. 7 illustrates a schematic diagram of a BAT system including one or more electrodes applied externally to stimulate baroreceptors without implantation, as constructed in accordance with at least one embodiment.

FIG. 8 illustrates a schematic diagram of a multi-channel BAT system that allows targeted baroreceptor activation with variable intensities, frequencies, and duty cycles, as constructed in accordance with at least one embodiment.

The drawings are not necessarily to scale. Certain features and components may be shown exaggerated in scale or in schematic form, and some details may not be shown in the interest of clarity and conciseness.

### DETAILED DESCRIPTION

The present disclosure describes systems and methods for using BAT to modulate ANS activity for treating inflammatory diseases, autoimmune disorders, cancer, chronic pain conditions, arrhythmias linked to inflammatory states, and Type II diabetes and metabolic dysfunction. BAT restores autonomic balance by reducing SNS overactivity and enhancing parasympathetic tone, thereby decreasing systemic inflammation and immune dysregulation.

### System Components and Operation

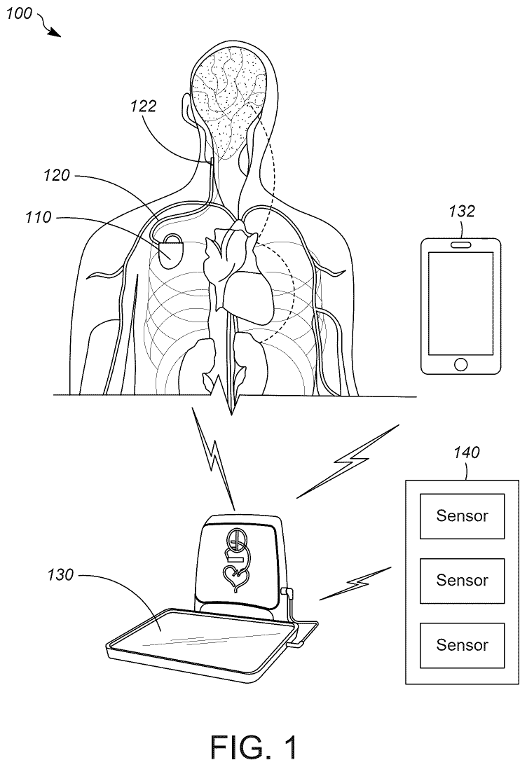

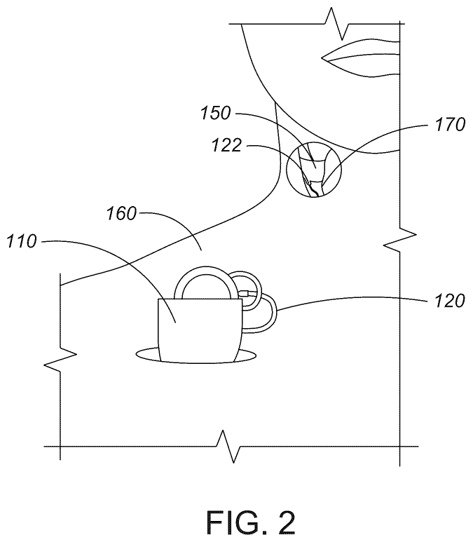

The BAT system (100) consists of several key components that work together to provide precise neuromodulation. As illustrated in FIGS. 1 and 2, the system can include an IPG (110), which is positioned subcutaneously near the clavicle (160), and a flexible lead (120) with one or more electrodes (122), placed along the carotid artery (170) to stimulate baroreceptors and regulate autonomic function. An external programmer (130) allows clinicians or industry professionals to adjust therapy settings, while feedback sensors (140) monitor heart rate variability (HRV), inflammatory biomarkers (e.g., TNF-α, IL-6, CRP), and electrophysiological activity to provide real-time feedback on autonomic and inflammatory status.

The BAT system (100) can offer advanced remote monitoring and therapy adjustment capabilities, enhancing patient care and convenience. Beyond physiological monitoring, the system can collect patient-reported symptoms, such as shortness of breath and swelling, through a dedicated interface (132) like a mobile app. This data can be transmitted to a central office for analysis, allowing for remote therapy adjustments without requiring in-person visits. Additionally, the system (100) can integrate heart failure management by utilizing physician-directed algorithms to assess both patient-reported symptoms and physiological data. This enables actionable interventions, including alerts prompting patients to contact a healthcare provider if symptoms indicate potential concerns and therapy modifications, such as adjustments to diuretic or insulin dosages based on real-time physiological feedback.

Patients can also initiate therapy updates through the interface (132). For example, pressing a designated button, such as a green button, may trigger the download of a new titration program, ensuring that therapy remains responsive to their evolving needs. To further enhance ease of use, the patient interface may be designed to be intuitive, incorporating simple feedback mechanisms such as smiley or sad face buttons. Selecting a smiley face maintains the current therapy settings, while selecting a sad face prompts an automatic therapy modification. The interface can also be customized based on specific patient conditions, including blood pressure, joint soreness, appetite, and urination. Each patient has access only to their designated screen, ensuring a personalized and user-friendly experience tailored to their individual needs.

The external programmer (130) serves as a central interface for clinicians and physicians, allowing real-time therapy monitoring and adjustments. The clinician/programmer interface can provide an advanced programming screen with protected access to prevent unauthorized modifications, with potential for expanded security and usability features. For physicians, a high-level dashboard can aggregate real-time patient data, generating an AI-driven patient health score for quick assessment. The system (100) can also include an alert feature, guiding physicians to a detailed evaluation and therapy adjustment screen, optimizing treatment pathways, and improving patient outcomes.

The BAT system provides therapeutic benefits by stimulating baroreceptors in the carotid sinus (150), for example, which reduces SNS hyperactivity, enhances vagal nerve signaling, and lowers pro-inflammatory cytokine production (e.g., TNF-α, IFN-γ, IL-1, IL-6, IL-12, CRP). This mechanism modulates immune function, suppresses chronic inflammation, and stabilizes electrophysiological activity, offering a non-pharmacological alternative for treating conditions driven by autonomic dysfunction and inflammation. While the carotid sinus (150) is a primary target, other baroreceptor sites, including the aortic arch, heart, and blood vessels, may also be stimulated. Additionally, central modulation techniques, such as oxytocin-based approaches, may further enhance therapeutic outcomes.

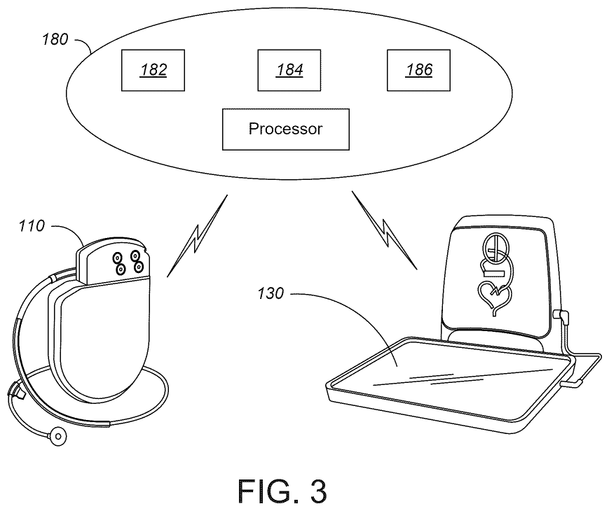

As depicted in FIG. 3, the BAT system (100) can include a closed-loop feedback module (180) that continuously monitors real-time physiological signals and autonomously adjusts therapy parameters. This module may include HRV sensors (182), biomarker sensors (184) for tracking inflammatory cytokines, and EMG acquisition systems (186) for detecting neuromuscular and autonomic function. Additionally, the module (180) may include blood pressure monitors. By leveraging continuous physiological monitoring, the closed-loop system dynamically modifies stimulation settings based on biomarker fluctuations and autonomic balance changes, ensuring adaptive neuromodulation tailored to the patient's evolving needs.

In some embodiments, the BAT system (100) incorporates AI-driven algorithms and machine learning models to analyze biometric trends, predict patient responses, and optimize neuromodulation settings in real time. By integrating automated therapy adjustments with clinician-, industry-, or patient-guided refinements, the system (100) provides a personalized and scalable solution for treating autonomic dysfunction, inflammatory diseases, and chronic pain conditions.

### Physiological Mechanism of Action

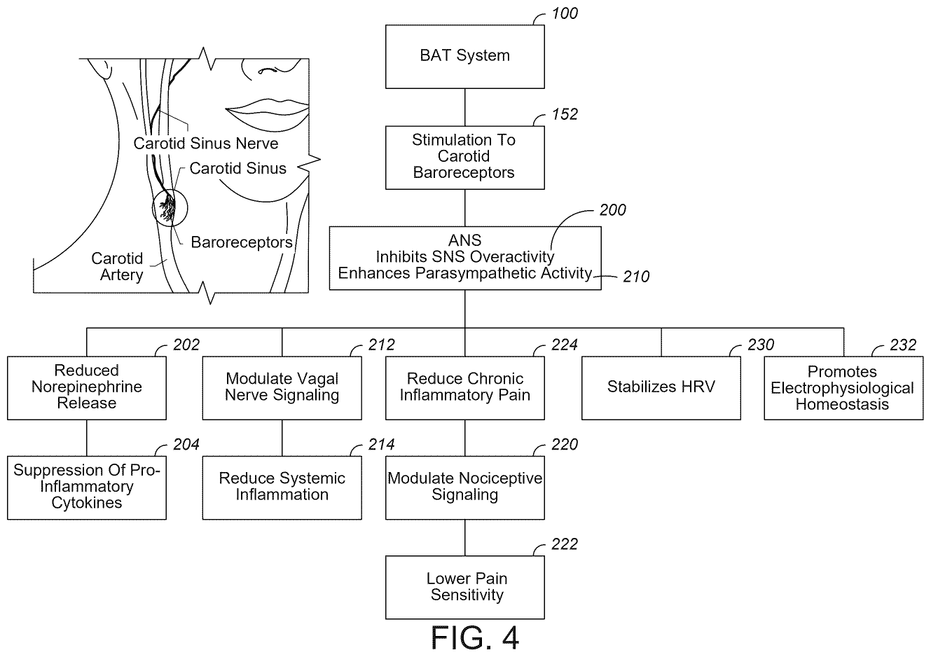

When activated, the BAT system (100) delivers controlled electrical stimulation to carotid baroreceptors (152), initiating a cascade of physiological responses that restore autonomic balance and modulate inflammatory and immune functions (FIG. 4). BAT inhibits SNS overactivity (200), leading to reduced norepinephrine release (202) and suppression of pro-inflammatory cytokines (204) such as IL-1, IL-6, IL-12, TNF-α, IFN-γ, and CRP.

Simultaneously, BAT enhances parasympathetic activity (210) by modulating vagal nerve signaling (212), which activates the cholinergic anti-inflammatory pathway to reduce systemic inflammation (214). This bidirectional autonomic modulation enables BAT to systemically regulate inflammatory responses, making it a promising therapeutic approach for immune-mediated diseases.

Beyond its impact on inflammation, BAT modulates nociceptive signaling (220) by reducing hyperalgesia and chronic inflammatory pain (224). By influencing nociceptive processing in the central and peripheral nervous systems, BAT lowers pain sensitivity (222), offering a non-opioid alternative for chronic pain management. Additionally, BAT is believed to modulate the patient's general pain reception, which can be quantified through patient questionnaires assessing pain perception and severity over time.

Additionally, BAT stabilizes HRV (230) and promotes electrophysiological homeostasis (232), maintaining cardiac autonomic stability and reducing arrhythmic risk. By addressing autonomic dysfunction and chronic inflammation, BAT provides a systemic, non-pharmacological approach to treating inflammatory diseases, autoimmune disorders, chronic pain, and cardiovascular dysfunction.

### Stimulation Protocols and Parameter Optimization

The BAT system (100) allows adjustable stimulation protocols to optimize therapeutic efficacy based on individual patient needs. The stimulation parameters can be fine-tuned to achieve precise neuromodulation, thereby enabling targeted autonomic and inflammatory modulation. Initial stimulation settings may include a pulse amplitude ranging from 0 -20 mA, a pulse width ranging from 0 -500 microseconds, a stimulation frequency ranging from 0 -100 Hz, and a duty cycle ranging from 20%-60% of active stimulation.

Various duty cycle configurations can be used, such as alternating between stimulation and rest periods or synchronizing therapy with the patient's sleep cycle or activity level. Additionally, duty cycle input variables can be customized to provide a highly personalized therapy experience. For example, time-of-day-based scheduling can be implemented such that therapy is activated at specific times, including turning on at 6 AM, turning off at 9 AM, and turning back on at 12 noon. Active therapy duration can also be controlled, with therapy delivered in defined time increments, such as one minute on and one minute off, or in longer or shorter cycles as clinically appropriate.

Day-of-the-week-based scheduling may be employed to follow a structured activation pattern, such as activating therapy on the first, third, fifth, and seventh days of the week while remaining off on the second, fourth, and sixth days. Activity level-based adjustments can be implemented using an accelerometer, allowing therapy intensity to be adjusted in response to physical activity. For example, therapy intensity can be reduced during sleep and increased when activity levels rise. Energy level modulation may also be performed, wherein energy output, defined as Amplitude×Pulse Width, is dynamically adjusted, such as operating at full power, half power, quarter power, or cycling through various power levels. In addition, therapy frequency can be modified in coordination with any of the foregoing variables to optimize neuromodulation.

Patient-controlled adjustments may further be provided, enabling patients to adjust therapy intensity, select predefined therapy programs, or modify stimulation settings based on comfort and clinical needs. These input variables can be combined in any number of configurations, allowing therapy to be highly personalized and adaptable. Duty cycling may also be tailored to specific clinical needs, such as increasing therapy intensity during cancer treatments or during periods of heightened autonomic dysfunction.

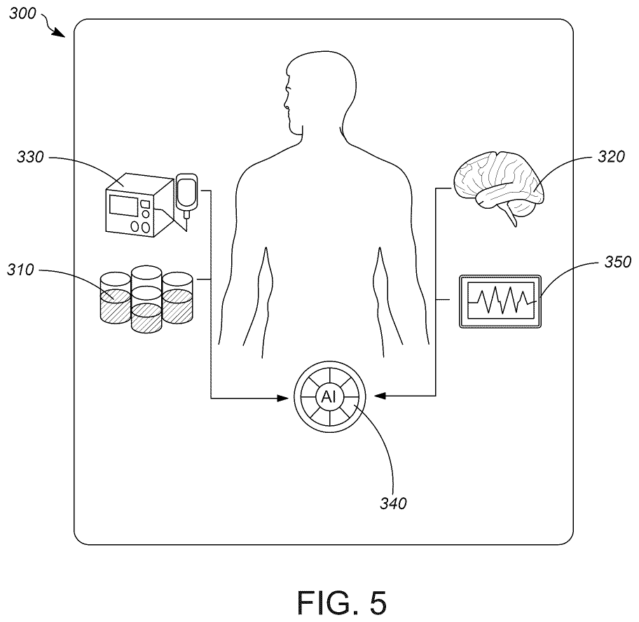

As illustrated in FIG. 5, the system may integrate AI-driven algorithms (300) for adaptive therapy optimization. These computational models analyze biomarker trends (310) to track inflammatory markers such as TNF-α, IL-1, IFN-γ, IL-6, IL-12, and CRP. The models further evaluate historical patient response patterns (320) to assess individualized therapy effectiveness over time and process real-time physiological feedback (330), including HRV, blood pressure, and EMG data, to enable continuous monitoring of autonomic function.

Additionally, machine learning models (340) can dynamically adjust therapy settings based on sensor data (350), refining stimulation parameters to align with patient-specific autonomic and immune responses. By leveraging real-time data processing, the BAT system (100) can optimize stimulation protocols for enhanced inflammation control, cardiovascular stability, and chronic pain management.

With AI-enhanced adaptability and closed-loop feedback, BAT represents a neuromodulation platform capable of delivering sustained, optimized therapeutic outcomes across a broad range of autonomic and inflammatory conditions.

### Implantation Procedure

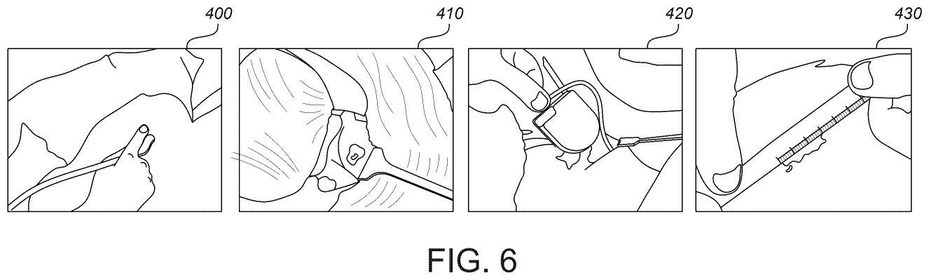

The implantation process for the BAT system (100) is designed to ensure precise baroreceptor activation while minimizing surgical complexity and optimizing long-term therapeutic outcomes. As illustrated in FIG. 6, the procedure may include a preparation step (400), during which the carotid sinus is localized using ultrasound guidance to ensure accurate lead placement and optimal baroreceptor stimulation. A lead placement step (410) may follow, in which a small incision is made near the sternocleidomastoid muscle to facilitate precise positioning of the lead. An IPG placement step (420) may then be performed, wherein a subcutaneous pocket is created, typically near the clavicle, to securely position the IPG while minimizing patient discomfort.

Following placement, a closure step (430) may be carried out, during which the IPG is secured and the incision is carefully closed to promote proper healing. Intraoperative adjustment may also be performed, with stimulation parameters fine-tuned during surgery to optimize baroreceptor activation and ensure balanced neuromodulation of the sympathetic and parasympathetic nervous systems. Postoperative monitoring and titration may then be conducted, during which therapy efficacy is assessed through ongoing monitoring and programmed titration, allowing refinement of stimulation settings to maximize long-term patient outcomes.

The BAT system can be designed to streamline the implantation process while ensuring effective autonomic modulation. Real-time intraoperative adjustments and postoperative therapy optimization allow precise, patient-specific stimulation settings that enhance therapeutic benefits while minimizing risks. Additionally, closed-loop feedback mechanisms and AI-driven therapy adjustments may be integrated to enable continuous, adaptive stimulation based on real-time physiological responses. This approach enhances clinical efficacy and supports long-term neuromodulation for inflammatory diseases, autoimmune disorders, cardiovascular conditions, and chronic pain management.

### Alternative Embodiments and Variations

Several alternative embodiments and variations of the BAT system may be implemented to enhance functionality, adaptability, and patient comfort, ensuring personalized therapy across diverse clinical applications.

In one embodiment, the BAT system features a wireless IPG, eliminating the need for lead wires, thereby reducing surgical complexity and improving patient comfort. This design enhances implantation feasibility and reduces the risk of lead-related complications, offering a more efficient and patient-friendly neuromodulation approach.

Another embodiment utilizes external neuromodulation approaches to provide temporary therapeutic relief without requiring implantation. This non-invasive option offers a flexible alternative for patients who may not require permanent implantation, including those experiencing acute inflammatory episodes, post-surgical recovery, or short-term autonomic modulation for cytokine storms.

Additionally, an embodiment integrates biosensors capable of real-time monitoring of inflammatory markers, autonomic function, and electrophysiological parameters. This enables a fully adaptive closed-loop therapy system, dynamically adjusting stimulation intensity, frequency, and duty cycle based on patient-specific physiological responses. By incorporating HRV analysis, inflammatory cytokine tracking (e.g., TNF-α, IFN-γ, IL-1, IL-6, IL-12, and CRP), and autonomic function assessments, the system can ensure precise and individualized therapy optimization.

Further variations include implantable BAT devices with multi-channel stimulation capabilities, allowing for targeted baroreceptor activation with variable stimulation intensities, frequencies, and duty cycles. This precision-driven stimulation approach can ensure that therapy is tailored for specific disease states, optimizing neuromodulation effectiveness for inflammatory, autoimmune, oncological, and cardiovascular conditions.

By integrating wireless communication, AI-driven closed-loop control, and non-invasive alternatives, the BAT system can provide a highly adaptable neuromodulation platform, offering therapeutic solutions for a broad range of autonomic and inflammation-related disorders.

### Wireless Bat System

An alternative embodiment features a wireless IPG that eliminates the need for lead wires, enhancing patient comfort, reducing surgical complexity, and minimizing the risk of lead-related complications. By eliminating traditional wiring, this wireless configuration allows for greater implantation flexibility, making it less invasive while maintaining optimal neuromodulation functionality.

This design can incorporate a wireless power transmission system to deliver energy to the IPG, ensuring continuous operation without requiring physical wiring or battery replacements. This advancement enhances long-term device sustainability and reduces the need for surgical interventions related to battery replacements or lead malfunctions.

Additionally, a data transmission module can facilitate seamless wireless communication between the IPG and an external programmer. This feature enables remote therapy adjustments, allowing clinicians or industry professionals to modify stimulation parameters, including intensity, frequency, and duty cycle, without requiring in-person device reprogramming. Such a wireless configuration provides greater flexibility in therapy customization, optimizing patient-specific neuromodulation responses in real time.

By integrating wireless power and data transmission technologies, this wireless BAT system significantly enhances patient convenience, device longevity, and therapeutic adaptability. The reduction in surgical complexity makes this system particularly beneficial for patients requiring long-term autonomic modulation for inflammatory, autoimmune, cardiovascular, and pain-related conditions.

### Non-Invasive Bat System

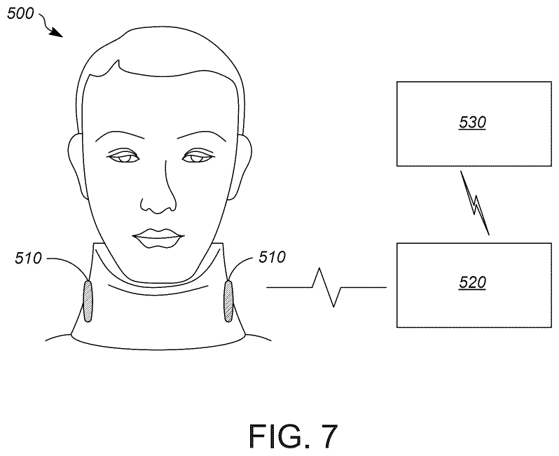

As illustrated in FIG. 7, another embodiment of the BAT system (500) features a non-invasive design that delivers electrical stimulation to the carotid sinus without requiring surgical implantation. This system utilizes external electrodes (510) placed near the carotid sinus, enabling targeted neuromodulation through the skin. This approach provides a less invasive alternative to implantable BAT systems.

A control unit (520) regulates stimulation settings and communicates wirelessly with an external programmer (530), enabling clinician-, industry-, or patient-guided therapy adjustments. This wireless communication feature allows for precise modulation of stimulation parameters, ensuring optimal neuromodulation without surgical intervention.

Additionally, feedback sensors can continuously monitor patient responses, tracking autonomic function, inflammatory markers, and cardiovascular parameters, allowing real-time therapy optimization. This adaptive feedback mechanism can ensure that stimulation parameters can be dynamically adjusted based on patient-specific physiological responses, improving both treatment efficacy and safety.

This non-invasive BAT system provides a flexible and temporary treatment option, making it particularly beneficial for patients who may not require permanent implantation or those who are not suitable candidates for surgery. It can be especially useful for short-term autonomic modulation, such as during acute inflammatory episodes, cytokine storms, perioperative autonomic stabilization, or post-surgical recovery. By offering a non-invasive, externally controlled alternative, this system broadens the accessibility and adaptability of BAT therapy, ensuring effective neuromodulation for a wider range of patient populations and clinical scenarios.

### Multi-Channel Stimulation Configuration

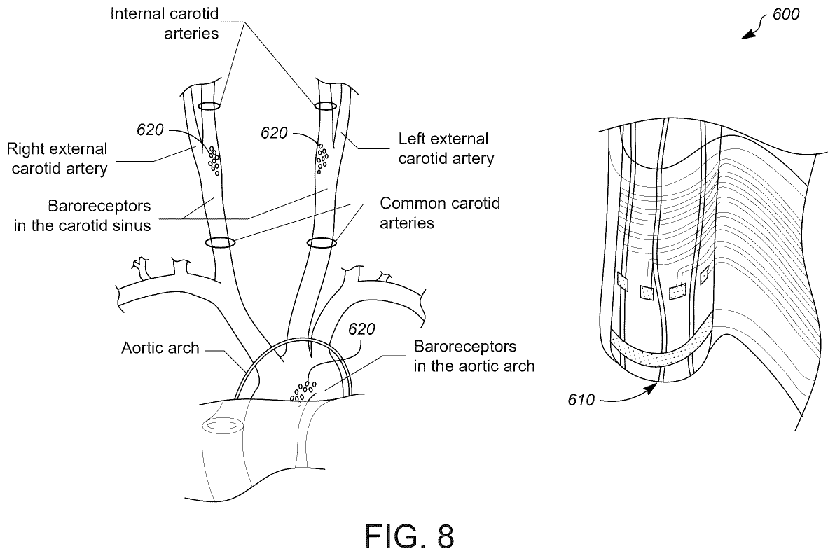

As illustrated in FIG. 8, a multi-channel BAT system (600) can be designed to enhance neuromodulation precision by enabling targeted baroreceptor stimulation. This system incorporates a multi-electrode lead (610), optionally in the form of a cuff, which connects to the IPG (110) (FIG. 1) to provide selective and adaptive stimulation across multiple baroreceptor sites (620). By allowing differentiated activation of distinct baroreceptor regions, this system enhances therapeutic specificity and autonomic regulation.

The multi-channel BAT system supports variable stimulation parameters, including adjustments to intensity, frequency, and duty cycle on a per-channel basis. This capability can enable customized therapy tailored to different physiological states and patient-specific autonomic responses, ensuring stimulation is optimized for maximum efficacy across various conditions.

By dynamically modulating multiple baroreceptor regions, the multi-channel configuration optimizes therapeutic outcomes, offering greater precision in autonomic regulation. This system is particularly beneficial for treating complex inflammatory, cardiovascular, and pain-related conditions, where autonomic dysfunction and systemic inflammation play a central role. The ability to fine-tune stimulation across multiple channels ensures that patients receive highly personalized therapy, improving long-term clinical outcomes while minimizing unwanted side effects and overstimulation risks.

### Wearable Interface and Remote Monitoring System

A wearable interface and remote monitoring system can enable real-time patient data tracking and therapy adjustments, thereby enhancing treatment personalization and long-term patient management. The system can incorporate implantable, external, or wearable devices designed to continuously monitor physiological parameters, including HRV, inflammatory biomarker levels such as TNF-α, IFN-γ, IL-1, IL-6, IL-12, and CRP, and electrophysiological activity. These real-time physiological metrics provide critical insights into autonomic function, inflammation levels, and cardiovascular stability, thereby enabling dynamic therapy optimization.

The system can establish wireless communication between the implantable, external, or wearable devices, the IPG, and an external programmer, ensuring seamless integration for data collection and closed-loop therapy adjustments. This connectivity can enable clinicians to remotely modify stimulation settings via a mobile application or a clinician dashboard, offering greater flexibility in patient management while reducing the need for frequent in-clinic visits.

Other advancements in BAT technology include the integration of machine learning algorithms to predict patient responses and automatically adjust stimulation settings based on historical data, biomarker trends, and real-time physiological feedback. By leveraging AI-driven analytics and adaptive therapy optimization, the system enhances long-term neuromodulation efficacy, autonomic balance, and inflammation control.

By integrating remote monitoring, real-time physiological tracking, AI-driven optimization, and wearable-based therapy control, this advanced BAT system represents the next generation of personalized neuromodulation therapies for a wide range of autonomic, inflammatory, and chronic disease conditions.

### Applications of Bat Therapy

The inventors believe that chronic sympathetic nervous system (SNS) overactivity contributes to systemic inflammation, cytokine dysregulation, and immune dysfunction, thereby influencing the progression of various disease states. These conditions may include inflammatory diseases such as rheumatoid arthritis (RA), systemic lupus erythematosus (SLE), and inflammatory bowel diseases, including Crohn's disease and ulcerative colitis, in which elevated levels of TNF-α, IL-1, IL-6, and IL-12 drive persistent immune activation. The inventors further believe that chronic SNS overactivity contributes to autoimmune disorders, in which excessive sympathetic signaling results in immune cell overactivation, cytokine imbalance, and sustained inflammation.

In addition, chronic sympathetic overactivity may promote cancer progression, as adrenergic signaling can enhance angiogenesis, immune evasion, and metastatic potential, thereby creating a tumor-supportive microenvironment. Cardiac arrhythmias linked to inflammation, including atrial fibrillation, myocarditis, and post-surgical arrhythmias, may also be influenced by inflammatory mediators that contribute to electrophysiological instability. Chronic inflammatory pain conditions, such as arthritis, joint pain, and neuropathic pain syndromes, may likewise be exacerbated by SNS overactivity, which modulates nociceptive signaling and pain sensitivity. Furthermore, Type II diabetes and metabolic dysfunction may be associated with autonomic dysregulation, reduced baroreflex sensitivity, and chronic inflammation, which together contribute to insulin resistance and pancreatic beta cell dysfunction.

### Chronic Inflammatory Diseases and Autoimmune Disorders

BAT therapy has significant potential in treating chronic inflammatory and autoimmune disorders, including RA, SLE, Crohn's disease, and ulcerative colitis. In RA, BAT can reduce joint inflammation and pain sensitivity by inhibiting sympathetic-driven cytokine release, specifically suppressing TNF-α and IL-6, key mediators of joint degradation. In SLE, BAT modulates immune activity, potentially preventing autoimmune flare-ups and reducing systemic inflammation.

For inflammatory bowel diseases, such as Crohn's disease and ulcerative colitis, BAT may attenuate gut-related neuroimmune interactions, reducing autonomic dysregulation and inflammatory damage. By restoring sympathovagal balance, BAT provides systemic inflammatory control, offering a non-pharmacological alternative or adjunct to immunosuppressants, biologic therapies (e.g., TNF inhibitors, IL-6 blockers), and corticosteroids. BAT's ability to regulate immune function makes it a promising strategy for improving long-term disease management while reducing reliance on traditional anti-inflammatory drugs.

### Oncology Applications

BAT has potential as an adjunctive therapy to inhibit tumor progression. Chronic sympathetic activation promotes tumor growth by stimulating angiogenesis, immune evasion, and metastasis, making autonomic modulation a promising therapeutic strategy. BAT may mitigate these effects by suppressing adrenergic-driven tumor progression, reducing tumor-associated inflammatory cytokines such as IL-6, TNF-α, and IFN-γ, and disrupting tumor microenvironment remodeling.

Additionally, BAT may enhance immune responses against malignant cells, potentially improving the efficacy of chemotherapy and immunotherapy by modulating the tumor-immune microenvironment. By balancing autonomic function and reducing tumor-supportive inflammation, BAT represents a novel neuromodulation-based strategy to complement existing cancer treatments, potentially improving patient outcomes and long-term survival. BAT therapy can be tailored to activate at specific times, such as before or after chemotherapy sessions, to optimize immune modulation.

### Cardiovascular Inflammation and Arrhythmias

BAT may offer significant benefits for cardiovascular inflammation and arrhythmias, particularly in conditions where chronic inflammation contributes to autonomic instability. Inflammatory processes play a central role in atherosclerotic plaque instability, increasing the risk of myocardial infarction and stroke. By downregulating inflammatory cytokines and restoring autonomic balance, BAT may help stabilize atherosclerotic plaques, reducing the likelihood of cardiovascular events.

Additionally, BAT, through improvements in HRV, may help reduce the occurrence of atrial fibrillation (AF) and ventricular tachyarrhythmias. The therapy may be particularly beneficial for patients with myocarditis, post-surgical inflammation-related AF, and autoimmune-associated arrhythmias, where inflammation contributes to electrophysiological instability. By addressing both autonomic dysfunction and inflammatory processes, BAT offers a novel approach to managing inflammation-driven cardiovascular disorders and reducing the risk of adverse cardiac events.

### Chronic Pain Management

BAT presents a non-opioid alternative for managing chronic inflammatory and neuropathic pain conditions, including arthritis, joint pain, neuropathy, and fibromyalgia. These conditions are often linked to maladaptive nociceptive signaling and neurogenic inflammation, both of which are exacerbated by SNS overactivity.

By modulating nociceptive pathways, BAT can reduce hyperalgesia and pain sensitivity, providing long-term pain relief without the risks of opioid dependency or tolerance. By restoring autonomic balance and reducing SNS-driven pain amplification, BAT offers a sustained, non-addictive neuromodulation-based therapy for patients with chronic inflammatory and neuropathic pain conditions.

### Type II Diabetes and Metabolic Dysfunction

BAT has potential as a novel therapeutic approach for Type II diabetes by addressing the underlying autonomic dysfunction and inflammation that contribute to metabolic disease. Reduced baroreflex sensitivity and chronic sympathetic overactivity are linked to insulin resistance, pancreatic beta cell dysfunction, and impaired glucose homeostasis.

High glucose levels stimulate the production of pro-inflammatory cytokines in pancreatic beta cells, leading to cell stress, apoptosis, and progressive loss of insulin secretion capacity. By reducing systemic inflammation and modulating autonomic tone, BAT may help preserve beta cell function, improve insulin sensitivity, and slow the progression of diabetes.

Additionally, BAT's ability to regulate inflammatory cytokines, such as IL-6 and TNF-α, may reduce the inflammatory burden associated with metabolic syndrome and obesity. By restoring autonomic homeostasis, BAT therapy offers a potential non-pharmacological strategy to complement existing diabetes treatments, improving glycemic control and reducing the risk of diabetes-related complications.

### Conclusion

The disclosed BAT systems and methods introduce a novel, adaptable, and effective approach to treating conditions driven by sympathetic overactivity and chronic inflammation. By leveraging neuromodulation as a systemic anti-inflammatory strategy, BAT extends beyond hypertension and heart failure applications into the broader regulation of immune responses and autonomic dysfunction. This positions BAT as a transformative therapy for a wide range of conditions, including inflammatory diseases, autoimmune disorders, cancer, chronic pain conditions, arrhythmias associated with inflammatory states, and Type II diabetes and metabolic dysfunction.

By providing a personalized, non-pharmacological therapy option, these BAT systems and methods reduce dependence on conventional drug therapies, which often come with adverse side effects and long-term tolerance issues. Instead, BAT offers a sustainable, long-term therapeutic alternative that targets the root causes of autonomic dysfunction and immune dysregulation rather than just managing symptoms.

Furthermore, the integration of adaptive neuromodulation, biosensor feedback, and machine learning-driven optimizations can ensure highly precise and individualized therapy. By continuously analyzing real-time physiological data, inflammatory markers, and autonomic function, BAT can dynamically adjust stimulation parameters to maximize therapeutic efficacy and patient safety. This makes BAT a scalable, patient-centric intervention uniquely suited to treating autonomic dysfunction and chronic inflammation-related conditions, ultimately improving clinical outcomes and quality of life for affected patients.

The above Detailed Description includes references to the accompanying drawings, which form a part of the Detailed Description. The Detailed Description should be read with reference to the drawings. The drawings show, by way of illustration, specific embodiments in which the present systems and methods can be practiced. These embodiments are also referred to herein as “examples.”

The above Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a method for regulating an inflammation or an immune response using baroreflex activation therapy comprises delivering electrical stimulation to baroreceptors. This stimulation modulates SNS activity while enhancing parasympathetic nervous system function. As a result, systemic inflammation is reduced by downregulating pro-inflammatory cytokines, contributing to improved immune regulation and autonomic balance.

In Example 2, the method of Example 1 is optionally configured such that delivering electrical stimulation to baroreceptors includes stimulating baroreceptors in the carotid sinus.

In Example 3, the method of any one of Examples 1 or 2 is optionally configured such that delivering electrical stimulation to baroreceptors includes applying a duty cycle that varies between 20% and 60% of active stimulation.

In Example 4, the method of any one of Examples 1-3 is optionally configured such that modulating sympathetic nervous system activity includes inhibiting norepinephrine release.

In Example 5, the method of any one of Examples 1-4 is optionally configured such that enhancing parasympathetic nervous system activity includes stimulating vagal nerve pathways.

In Example 6, the method of any one of Examples 1-5 is optionally configured such that downregulating pro-inflammatory cytokines includes reducing levels of interleukin-1 (IL-1), interleukin-6 (IL-6), interleukin-12 (IL-12), tumor necrosis factor-alpha (TNF-α), or C-reactive protein (CRP).

In Example 7, the method of any one of Examples 1-6 optionally further comprises dynamically adjusting one or more stimulation parameters based on measured patient-specific responses.

In Example 8, the method of Example 7 is optionally configured such that the one or more stimulation parameters are modulated in response to changes in patient data detected by an implantable, wearable, or external sensor. The patient data comprises heart rate variability, blood pressure, biomarker levels, or electrophysiological activity.

In Example 9, the method of Example 8 is optionally configured such that modulating the one or more stimulation parameters includes analyzing real-time patient data using an artificial intelligence processing unit to determine optimal stimulation parameters.

In Example 10, the method of any one of Examples 1-9 is optionally configured such that the therapy is used to treat a chronic inflammatory disorder.

In Example 11, the method of any one of Examples 1-9 is optionally configured such that the therapy is used to treat an autoimmune disease.

In Example 12, the method of any one of Examples 1-9 is optionally configured such that the therapy is used to treat cancer progression.

In Example 13, the method of Example 12 is optionally configured such that the therapy is used as an adjunct to chemotherapy or immunotherapy to suppress adrenergic-driven tumor progression.

In Example 14, the method of any one of Examples 1-9 is optionally configured such that the therapy is used to treat neuropathic pain.

In Example 15, a method for treating arrhythmias associated with an inflammatory state using baroreflex activation therapy comprises applying electrical stimulation to baroreceptors via a pulse generator, monitoring heart rate variability and inflammatory biomarkers, and adjusting stimulation intensity, frequency, or duty cycle based on real-time physiological data. The method reduces autonomic nervous system dysregulation by modulating sympathetic and parasympathetic tone, thereby improving electrophysiological stability and decreasing arrhythmic risk.

In Example 16, the method of Example 15 optionally further comprises adjusting the stimulation frequency to improve heart rate variability and reduce the risk of atrial fibrillation.

In Example 17, the method of any one of Examples 15 or 16 is optionally further configured such that the therapy reduces the risk of myocardial infarction by stabilizing atherosclerotic plaques.

In Example 18, the method of any one of Examples 15-17 is optionally configured such that the therapy is applied to a patient with myocarditis or post-surgical inflammation-related atrial fibrillation.

In Example 19, a system for regulating an inflammation or immune response using baroreflex activation therapy comprises an implantable pulse generator, a flexible lead with at least one electrode configured to stimulate baroreceptors, and a feedback module. The feedback module comprises at least one sensor selected from inflammatory biomarker sensors, heart rate variability monitors, blood pressure monitors, and electromyography data acquisition systems. The system dynamically adjusts stimulation parameters based on real-time physiological feedback.

In Example 20, the system of Example 19 optionally further comprises a closed-loop controller that automatically adjusts stimulation based on detected inflammatory biomarker levels.

In Example 21, the system of any one of Examples 19 or 20 optionally further comprises an external programmer configured to adjust stimulation parameters.

In Example 22, the system of any of Examples 19-21 optionally further comprises a patient interface configured to receive patient-reported symptoms and inputs. The system integrates this patient-reported data with real-time physiological data to optimize stimulation parameters.

In Example 23, the system of Example 22 is optionally configured such that the patient interface is configured to facilitate remote therapy adjustments by transmitting collected data to an external programmer, enabling clinician-approved modifications to stimulation parameters without requiring an in-person hospital or clinic visit.

In Example 24, the system of any one of Examples 19-23 optionally further comprises an artificial intelligence-driven algorithm configured to predict patient response trends and adjust therapy accordingly.

In Example 25, the system of any one of Examples 19-24 optionally further comprises an external pulse generator that provides temporary stimulation prior to or instead of implantation.

In Example 26, the system of any one of Examples 19-25 is optionally configured such that the flexible lead comprises multiple stimulation channels configured for targeted baroreceptor activation.

In Example 27, a system for regulating inflammation and immune responses using baroreflex activation therapy comprises a pulse generator configured to deliver electrical stimulation to baroreceptors, at least one sensor configured to collect real-time physiological data related to autonomic function, inflammatory markers, or cardiovascular status, and a closed-loop control system configured to dynamically modify stimulation parameters based on the real-time physiological data. Additionally, the system features an artificial intelligence processing unit configured to analyze sensor data, predict patient responses, and optimize stimulation settings.

In Example 28, the system of Example 27 optionally further comprises an external programmer configured to receive and transmit control signals adjusting stimulation parameters.

In Example 29, the system of Example 28 optionally further comprises a patient interface configured to receive patient-reported symptoms and inputs. The artificial intelligence processing unit integrates this patient-reported data with real-time physiological data to optimize stimulation settings.

In Example 30, the system of Example 29 is optionally configured such that the pulse generator, at least one sensor, closed-loop control system, external programmer, patient interface, and artificial intelligence processing unit are configured to exchange data wirelessly, enabling real-time therapy adaptation.

In Example 31, the system of any one of Examples 29 or 30 optionally further comprises a wireless communication module enabling bidirectional data exchange between the pulse generator, at least one sensor, closed-loop control system, external programmer, patient interface, and artificial intelligence processing unit.

In Example 32, the system of any one of Examples 27-31 is optionally configured such that the pulse generator is an implantable pulse generator.

In Example 33, the system of any one of Examples 27-31 is optionally configured such that the pulse generator is an external stimulation unit.

In Example 34, the system of any one of Examples 27-33 optionally further comprises a multi-channel stimulation system configured to provide independent and adjustable stimulation intensities, frequencies, and duty cycles across multiple baroreceptor regions.

### Definitions and Interpretations

Throughout this patent document, specific terms are used to refer to particular features, components, and method steps. As one skilled in the art will recognize, different terminology may be used to describe the same or similar elements. The terminology used herein is not intended to limit the scope of the invention to any particular nomenclature. Instead, the terms should be understood in accordance with their function within the systems and methods described.

Unless explicitly defined otherwise within this document, the following definitions shall apply. The terms “a,” “an,” and “the” refer to one or more than one, irrespective of whether the phrase “at least one” or “one or more” is used in other contexts. The term “or” should be interpreted as a nonexclusive disjunction, meaning that “A or B” includes (i) A but not B, (ii) B but not A, and (iii) both A and B.

All numerical values provided in this disclosure should be understood as approximate values, whether explicitly stated or not, and are assumed to be modified by the term “about.” The term “about” encompasses numerical variations that are considered functionally equivalent by those skilled in the art. This may include rounding to the nearest significant figure or values within an equivalent range. Additionally, when numerical ranges are provided, the endpoints are inclusive of all intermediate values and subranges. For example, the range 1 to 4 includes 1, 1.5, 1.75, 2, 2.3, 2.6, 2.9, etc., as well as subranges such as 1 to 1.5, 1 to 2, 1 to 3, 2 to 3.5, 2 to 4, 3 to 4, etc.

### Scope and Claim Construction

The scope of the present systems and methods should be determined with reference to the appended claims, along with the full scope of equivalents to which such claims are entitled. In the claims, the term “including” is to be interpreted as the plain-English equivalents of “comprising” and “wherein.” The use of “including” and “comprising” in a claim should be understood as open-ended terminology, meaning that a system or method that includes additional features or components beyond those listed still falls within the scope of the claim.

### Abstract Interpretation

The Abstract of this application is provided solely for the purpose of enabling a quick understanding of the technical disclosure. It is submitted with the express understanding that it will not be used to interpret or limit the scope or meaning of the claims. The present systems and methods should be construed based on the full scope of the Detailed Description and Claim sections, rather than any limitations inferred from the Abstract.

1. A method for regulating inflammation in a patient using baroreflex activation therapy, comprising:
delivering electrical stimulation to one or more baroreceptors to reduce sympathetic nervous system overactivity and enhance parasympathetic activity;
monitoring real-time physiological signals comprising: (i) at least one inflammatory biomarker selected from TNF-α, IFN-γ, IL-1, IL-6, IL-12, and C-reactive protein (CRP); and (ii) at least one autonomic parameter comprising heart rate variability (HRV), blood pressure, or electromyography (EMG) data;
analyzing biomarker fluctuations and autonomic balance changes; and
dynamically modifying one or more stimulation parameters based on the biomarker fluctuations and autonomic balance changes to suppress systemic inflammation.

2. The method of claim 1, wherein delivering electrical stimulation comprises stimulating baroreceptors in a carotid sinus.

3. The method of claim 1, wherein dynamically modifying the one or more stimulation parameters comprises adjusting stimulation amplitude, pulse width, frequency, or duty cycle.

4. The method of claim 3, wherein the duty cycle varies between 20% and 60% of active stimulation.

5. The method of claim 1, wherein analyzing biomarker fluctuations comprises analyzing biomarker trends over time.

6. The method of claim 1, wherein dynamically modifying the one or more stimulation parameters comprises executing AI-driven algorithms or machine learning models configured to analyze historical patient response patterns and real-time physiological feedback.

7. The method of claim 1, wherein the inflammation is associated with a chronic inflammatory disease or an autoimmune disorder.

8. The method of claim 1, wherein the inflammation is associated with cancer progression and stimulation is increased during a cancer treatment period.

9. The method of claim 1, wherein the inflammation is associated with a chronic inflammatory pain condition.

10. A baroreflex activation therapy system for modulating inflammation, comprising:
a pulse generator configured to deliver electrical stimulation to one or more baroreceptors;
a lead configured for targeted baroreceptor activation; and
a closed-loop feedback module comprising:
(i) at least one inflammatory biomarker sensor configured to detect TNF-α, IFN-γ, IL-1, IL-6, IL-12, or C-reactive protein (CRP);
(ii) at least one autonomic sensor comprising a heart rate variability (HRV) monitor, a blood pressure monitor, or an electromyography (EMG) acquisition system; and
(iii) a controller configured to: analyze biomarker fluctuations and autonomic balance changes; and dynamically modify stimulation amplitude, pulse width, frequency, or duty cycle based on the biomarker fluctuations and autonomic balance changes.

11. The system of claim 10, wherein the pulse generator comprises an implantable pulse generator.

12. The system of claim 10, wherein the pulse generator comprises an external stimulation unit.

13. The system of claim 10, further comprising an external programmer configured to adjust stimulation parameters.

14. The system of claim 10, further comprising a patient interface configured to receive patient-reported symptoms and integrate the patient-reported symptoms with real-time physiological data to optimize stimulation parameters.

15. The system of claim 10, further comprising a wireless communication module enabling bidirectional data exchange between the pulse generator, the closed-loop feedback module, and an external programmer.

16. The system of claim 10, wherein the controller comprises AI-driven algorithms or machine learning models configured to analyze biomarker trends and predict patient responses.

17. The system of claim 10, wherein the controller analyzes real-time physiological signals from the inflammatory biomarker sensor and the autonomic sensor.

18. The system of claim 10, wherein the lead comprises multiple independently controllable stimulation channels configured for selective activation of distinct baroreceptor regions.

19. A method for treating an arrhythmia associated with inflammation using baroreflex activation therapy, comprising:
delivering electrical stimulation to one or more baroreceptors;
monitoring heart rate variability (HRV) and at least one inflammatory biomarker selected from TNF-α, IFN-γ, IL-1, IL-6, IL-12, and C-reactive protein (CRP);
analyzing changes in HRV and biomarker fluctuations; and
dynamically adjusting stimulation frequency based on the analyzed changes to improve electrophysiological stability and decrease arrhythmic risk.

20. The method of claim 19, wherein the arrhythmia comprises atrial fibrillation.

21. The method of claim 19, wherein the arrhythmia is associated with myocarditis or post-surgical inflammation-related atrial fibrillation.

22. The method of claim 19, wherein dynamically adjusting stimulation frequency increases heart rate variability.
